(19) United States
(12) Patent Application Publication
(10) Pub. No.: US 2018/0078236 A1
(43) Pub. Date: Mar. 22, 2018

(54) DIAGNOSIS SYSTEM AND METHOD USING PHOTOACOUSTIC/ULTRASOUND CONTRAST AGENT

(71) Applicant: SOGANG UNIVERSITY RESEARCH FOUNDATION, Seoul (KR)

(72) Inventors: Hyuncheol KIM, Seoul (KR); Jin Ho CHANG, Seoul (KR); Hyung Won MOON, Seoul (KR); Ho Hyun LEE, Seoul (KR)

(21) Appl. No.: 15/560,980

(22) PCT Filed: Oct. 14, 2015
(86) PCT No.: PCT/KR2015/010803

§ 371 Date: Sep. 22, 2017

(30) Foreign Application Priority Data
Apr. 15, 2015 (KR) 10-2015-0053327

(51) Int. Cl. A61B 8/00; A61B 5/00; A61B 8/08; A61K 49/22

(52) U.S. Cl. CPC A61B 8/4416; A61B 5/0095; A61K 49/223; A61B 8/4477; A61B 8/481

(57) ABSTRACT

The present invention relates to a diagnosis system and method using a contrast agent used in photoacoustic image diagnosis and ultrasound image diagnosis. More particularly, the present invention relates to a diagnosis system and method using a photoacoustic/ultrasound contrast agent, the system and method discriminating and selectively detecting ultrasonic waves emitted from respective contrast agents by analyzing a correlation between a size of a contrast agent and a frequency of an ultrasonic wave emitted from the contrast agent, thereby obtaining a plurality of clear photoacoustic images.

### CROSS-REFERENCE TO RELATED APPLICATIONS

This application claims the benefit under 35 U.S.C. section 371, of PCT International Application No.: PCT/KR/KR2015/010803, filed on Oct. 14, 2015, which claims foreign priority to Korean Patent Application No.: KR10-2015-0053327, filed on Apr. 15, 2015, in the Korean Intellectual Property Office, both of which are hereby incorporated by reference in their entireties.

### TECHNICAL FIELD

The present invention relates to a diagnosis system and method using a contrast agent used in photoacoustic image diagnosis and ultrasound image diagnosis. More particularly, the present invention relates to a diagnosis system and method using a photoacoustic/ultrasound contrast agent, the system and method discriminating and selectively detecting ultrasonic waves emitted from respective contrast agents by analyzing a correlation between a size of a contrast agent and a frequency of an ultrasonic wave emitted from the contrast agent, thereby obtaining a plurality of clear photoacoustic images.

### BACKGROUND ART

Accurate imaging of biological targets is an important tool for diagnosing various diseases without error. Recently, in order to overcome the drawbacks of a single imaging modality, a multi-mode imaging technology that is a combination of imaging technologies of different modalities has been developed. As an example of the multi-mode imaging technology, there is a combination of photoacoustic image diagnosis where light is emitted into a body and ultrasound emitted from the body is detected and imaged, and ultrasound image diagnosis where ultrasound is applied into a body and emitted ultrasound is detected and imaged. To be used in the photoacoustic image diagnosis and the ultrasound image diagnosis, a photoacoustic/ultrasound contrast agent enabling clear diagnosis to be performed by providing artificial contrast effect to the affected part has been developed like the following patent document.

Patent Document

Korean Patent Application Publication No. 10-2015-0010908 (29 Jan. 2015) “CONTRAST AGENT FOR COMBINED PHOTOACOUSTIC AND ULTRASOUND IMAGING”

However, a diagnosis method using a conventional photoacoustic/ultrasound contrast agent is problematic in that clear photoacoustic images and multi-images cannot be obtained. Therefore, there is a need for a technology of obtaining clear photoacoustic images and multi-images by analyzing a correlation between a contrast agent and an ultrasonic wave emitted from the contrast agent.

### DISCLOSURE

### Technical Problem

Accordingly, the present invention has been made keeping in mind the above problems occurring in the related art.

An object of the present invention is to provide a diagnosis system and method using a photoacoustic/ultrasound contrast agent, the system and method being capable of performing clear photoacoustic diagnosis by obtaining a photoacoustic image based on a correlation between the size of the contrast agent and a frequency of an ultrasonic wave emitted from the contrast agent.

Also, an object of the present invention is to provide a diagnosis system and method using a photoacoustic/ultrasound contrast agent, the system and method being capable of obtaining a plurality of photoacoustic images by using a plurality of contrast agents having different sizes and by selectively detecting ultrasonic waves emitted from respective contrast agents.

### Technical Solution

In order to accomplish the above object, the present invention is realized by embodiments having the following configurations.

According to an embodiment of the present invention, a diagnosis method using a photoacoustic/ultrasound contrast agent includes obtaining a photoacoustic image by considering a correlation between a size of a photoacoustic/ultrasound contrast agent and a frequency of an ultrasonic wave that is a photoacoustic signal emitted from the contrast agent.

According to another embodiment of the present invention, the diagnosis method may further include: emitting, at a photoacoustic signal generation step, light to the photoacoustic/ultrasound contrast agent such that the ultrasonic wave having a particular frequency is emitted; and detecting, at a detection imaging step, the ultrasonic wave emitted at the photoacoustic signal generation step and imaging the ultrasonic wave, wherein at the detection imaging step, the frequency of the ultrasonic wave that varies depending on the size of the photoacoustic/ultrasound contrast agent is selectively detected and imaged.

According to still another embodiment of the present invention, at the photoacoustic signal generation step, a plurality of photoacoustic/ultrasound contrast agents having different sizes may be used such that a plurality of ultrasonic waves having different frequencies is emitted, and at the detection imaging step, the plurality of ultrasonic waves having the different frequencies may be detected and imaged such that a plurality of photoacoustic images is obtained.

According to still another embodiment of the present invention, the plurality of photoacoustic/ultrasound contrast agents may be injected into different parts in a body, and at the detection imaging step, the plurality of photoacoustic images of the different parts may be simultaneously obtained.

According to still another embodiment of the present invention, the plurality of photoacoustic/ultrasound contrast agents may be injected into a same part in a body, and at the detection imaging step, the plurality of photoacoustic images of the same part may be obtained.

According to still another embodiment of the present invention, the plurality of photoacoustic/ultrasound contrast agents may target different biomarkers such that the plurality of photoacoustic images is obtained at the detection imaging step, whereby multi-imaging is performed.

According to still another embodiment of the present invention, each of the plurality of photoacoustic/ultrasound contrast agents may include a ligand that is conjugated to a surface thereof and targets a particular biomarker.

According to still another embodiment of the present invention, at the detection imaging step, the plurality of photoacoustic images may have different colors.

According to still another embodiment of the present invention, a diagnosis system using a photoacoustic/ultrasound contrast agent obtains a photoacoustic image by considering a correlation between a size of a photoacoustic/ultrasound contrast agent and a frequency of an ultrasonic wave that is a photoacoustic signal emitted from the contrast agent.

According to still another embodiment of the present invention, the diagnosis system may further include: the photoacoustic/ultrasound contrast agent being injected into a body and being used as a photoacoustic and ultrasound contrast agent; a light emitting device emitting light to the photoacoustic/ultrasound contrast agent; and a detection imaging device detecting the ultrasonic wave, emitted from the photoacoustic/ultrasound contrast agent by absorbing the light of the light emitting device, and imaging the ultrasonic wave, wherein the detection imaging device selectively detects the frequency of the ultrasonic wave that varies depending on the size of the photoacoustic/ultrasound contrast agent so as to image the frequency.

According to still another embodiment of the present invention, the detection imaging device may include an ultrasound transducer detecting the ultrasonic wave emitted from the photoacoustic/ultrasound contrast agent, and the ultrasound transducer may have a detection frequency band containing the frequency of the ultrasonic wave emitted from a particular contrast agent.

According to still another embodiment of the present invention, a plurality of photoacoustic/ultrasound contrast agents having different sizes may be used such that frequencies of ultrasonic waves emitted from the plurality of photoacoustic/ultrasound contrast agents are different, and the detection imaging device may obtain a plurality of photoacoustic images by individually detecting and imaging the different frequencies of the ultrasonic waves.

According to still another embodiment of the present invention, the detection imaging device may include a plurality of ultrasound transducers detecting the ultrasonic waves emitted from the plurality of photoacoustic/ultrasound contrast agents, and each of the plurality of ultrasound transducers may have a frequency band containing the frequency of the ultrasonic wave emitted from the associated contrast agent.

According to still another embodiment of the present invention, as the ultrasound transducer, one wideband ultrasound transducer having all frequency bands of the ultrasonic waves emitted from the plurality of photoacoustic/ultrasound contrast agents may be used, or an integrated ultrasound transducer having different frequency bands may be used.

According to still another embodiment of the present invention, the photoacoustic/ultrasound contrast agent may include micro-bubbles having gas inside thereof and a photoacoustic contrast component that is conjugated to a surface of the micro-bubbles or is stacked inside thereof.

According to still another embodiment of the present invention, the photoacoustic/ultrasound contrast agent may include a ligand that is conjugated to the surface of the micro-bubbles and targets a particular biomarker, the gas may be at least one of perfluorocarbon and sulfur hexafluoride, the micro-bubbles may be made of protein or lipid, and the photoacoustic contrast component may be at least one of porphyrin, indocyanine green, green fluorescence protein (GFP), ferritin, and gold nanorod.

According to still another embodiment of the present invention, the frequency of the ultrasonic wave emitted from the photoacoustic/ultrasound contrast agent may be a resonant frequency that occurs when the ultrasonic wave emitted from the photoacoustic contrast component is resonated by the micro-bubble.

### Advantageous Effects

According to the embodiment of the present invention, various effects as follow may be obtained.

The present invention can obtain a photoacoustic image based on a correlation between a size of a contrast agent and a frequency of an ultrasonic wave emitted from the contrast agent, whereby clear photoacoustic diagnosis can be performed.

Also, the present invention can obtain a plurality of photoacoustic images by using a plurality of contrast agents having different sizes and by selectively detecting ultrasonic waves emitted from respective contrast agents.

### DESCRIPTION OF DRAWINGS

The patent or application file contains at least one drawing executed in color. Copies of this patent or patent application publication with color drawing(s) will be provided by the office upon request and payment of the necessary fee.

FIG. 1 is a view illustrating size distributions of samples 1 to 4.

FIG. 2 is a view illustrating detection frequency band distribution of each ultrasound transducer and intensity of the optimum frequency and signal according to a size of a photoacoustic/ultrasound contrast agent.

FIG. 3 is a photoacoustic image obtained by using samples 1 to 4.

FIG. 4 is a view illustrating intensity of quantitative photoacoustic signals in respective ultrasound transducers according to samples 1 to 4.

FIG. 5 is a view illustrating a relation between a size of a photoacoustic/ultrasound contrast agent and a resonant frequency in terms of theory and experiment.

### BEST MODE

Hereinafter, a diagnosis system and method using a photoacoustic/ultrasound contrast agent according to the present invention will be described in detail with reference to the accompanying drawings. Unless otherwise defined, all terms including technical and scientific terms used herein have the same meaning as commonly understood by one of ordinary skill in the art to which this invention belongs. When terms used herein discord from the commonly understood meaning, the terms will be interpreted as defined herein. Also, in the following description of the present invention, detailed descriptions of known functions and components incorporated herein will be omitted when it may make the subject matter of the present invention unclear. Also, it should also be understood that when a component “includes” an element, unless there is another opposite description thereto, the component does not exclude another element but may further include the other element.

An embodiment of the present invention relates to a diagnosis system using a photoacoustic/ultrasound contrast agent, the system obtaining a photoacoustic image based on a correlation between a size of a photoacoustic/ultrasound contrast agent and a frequency of an ultrasonic wave that is a photoacoustic signal emitted from the contrast agent. The diagnosis system includes: the photoacoustic/ultrasound contrast agent being injected into a body and being used as a photoacoustic and ultrasound contrast agent; a light emitting device (not shown) emitting light to the photoacoustic/ultrasound contrast agent; and a detection imaging device (not shown) detecting the ultrasonic wave, emitted from the photoacoustic/ultrasound contrast agent by absorbing the light of the light emitting device, and imaging the ultrasonic wave. The detection imaging device selectively detects the frequency of the ultrasonic wave that varies depending on the size of the photoacoustic/ultrasound contrast agent and emitted from the photoacoustic/ultrasound contrast agent so as to image the frequency.

The photoacoustic/ultrasound contrast agent is injected into a body and used as a photoacoustic and ultrasound contrast agent, and a conventional photoacoustic/ultrasound contrast agent may be used. However, preferably, the photoacoustic/ultrasound contrast agent includes micro-bubbles having gas inside thereof and a photoacoustic contrast component that is conjugated to a surface of the micro-bubbles or is stacked inside thereof. The gas is perfluorocarbon or/and sulfur hexafluoride, and the micro-bubbles are made of protein or lipid, and the photoacoustic contrast component is a substance that serves as a photoacoustic contrast agent such as porphyrin, indocyanine green, green fluorescence protein (GFP), ferritin, gold nanorod, etc. According to the present invention, a frequency of an ultrasonic wave that is a photoacoustic signal emitted from the contrast agent differs depending on the size of the photoacoustic/ultrasound contrast agent (i.e., the size of the micro-bubble). By using a plurality of contrast agents having different sizes, ultrasonic waves having different frequencies are emitted from the contrast agents. The ultrasonic waves having different frequencies are individually detected and imaged, thereby obtaining clear multi-images. Emitted frequency differs depending on the size of the contrast agent, since the ultrasonic wave emitted from the photoacoustic contrast component is resonated by the micro-bubbles and the ultrasonic wave having a particular frequency is emitted from the contrast agent. A plurality of photoacoustic/ultrasound contrast agents having different sizes may be injected into the same part in a body, or into different parts in the body. When obtaining a plurality of images by injecting the contrast agents into the same part, a plurality of images of one part may be obtained. When obtaining a plurality of images by injecting the contrast agents into different parts, a plurality of images of different parts may be simultaneously obtained.

The photoacoustic/ultrasound contrast agent may include a ligand that is conjugated to the surface of the micro-bubbles and targets a particular biomarker. For example, two photoacoustic/ultrasound contrast agents 1 (a ligand A targeting a particular biomarker a is conjugated to a surface of the contrast agent 1) and 2 (a ligand B targeting a particular biomarker b is conjugated to a surface of the contrast agent 2) having different sizes are prepared. When injecting contrast agents 1 and 2 into a body, the contrast agent 1 targets the biomarker a and conjugates the biomarker a thereto, and the contrast agent 2 targets the biomarker b and conjugates the biomarker b thereto.

The light emitting device emits light to the photoacoustic/ultrasound contrast agent injected into a body and, for example, the light has a wavelength of 700-800 nm that may be optimally absorbed by various photoacoustic contrast agents including porphyrin, which is a photoacoustic contrast component. For example, when emitting light to the contrast agents 1 and 2 respectively conjugated to the biomarkers a and b by using the light emitting device, the contrast agents 1 and 2 respectively emit ultrasonic waves having different frequencies.

The detection imaging device detects the ultrasonic wave, emitted from the photoacoustic/ultrasound contrast agent by absorbing the light of the light emitting device, and images the ultrasonic wave. The frequency of the ultrasonic wave, which varies depending on the size of the photoacoustic/ultrasound contrast agent and is emitted from the photoacoustic/ultrasound contrast agent, is selectively detected and imaged. The detection imaging device may include an ultrasound transducer, an ultrasound imaging device, etc.

The ultrasound transducer is a device detecting the ultrasonic wave emitted from the photoacoustic/ultrasound contrast agent and having a different detection frequency band. For example, ultrasound transducers C5-2/60, L14-5/38, and L40-8/12 on the market may respectively detect frequency bands of 1.8-5.1, 4.0-11.0, and 9.6-20.2 MHz, which will be described in the following embodiment. Depending on the size of the photoacoustic/ultrasound contrast agent, the frequency of the emitted ultrasonic wave differs. Thus, a clearer image may be obtained by using an ultrasound transducer having a frequency band containing the frequency of the ultrasonic wave emitted from a particular contrast agent. Also, when using a plurality of photoacoustic/ultrasound contrast agents having different sizes, ultrasound transducers are provided according to the number of the contrast agents, and clearer multi-images may be obtained by using the ultrasound transducers having frequency bands containing frequencies of ultrasonic waves emitted from respective contrast agents. For example, when a contrast agent emitting an ultrasonic wave having the optimum frequency of 8 MHz and a contrast agent emitting an ultrasonic wave having the optimum frequency of 15 MHz are used and the ultrasonic waves are detected by using the ultrasound transducers L14-5/38 and L40-8/12, two clear images may be obtained. Furthermore, one wideband ultrasound transducer having all frequency bands of the ultrasonic waves emitted from the photoacoustic/ultrasound contrast agents may be used, or an integrated ultrasound transducer having different frequency bands may be used. Consequently, all frequency bands of the ultrasonic waves of the photoacoustic/ultrasound contrast agents may be simultaneously obtained.

The ultrasound imaging device displays the photoacoustic image based on data detected and output by the ultrasound transducer. When ultrasonic waves having different frequencies are emitted by using a plurality of contrast agents having different sizes and a plurality of pieces of data is output by the ultrasound transducers, a plurality of photoacoustic images according to the number of the contrast agents may be obtained. Also, the ultrasound imaging device may display the plurality of photoacoustic images by respectively assigning different colors thereto. For example, when emitting light to the contrast agents 1 and 2 respectively conjugated to the biomarkers a and b by using the light emitting device so as to enable the contrast agents 1 and 2 to respectively emit ultrasonic waves having different frequencies, the ultrasound transducers detect respective ultrasonic waves and output a plurality of pieces of data. The ultrasound imaging device obtains a plurality of photoacoustic images having different colors based on the data. Consequently, distributions of the biomarkers a and b in a body can be identified, and multi-imaging is possible.

Another embodiment of the present invention relates to a diagnosis method using a photoacoustic/ultrasound contrast agent, the method obtaining a photoacoustic image based on a correlation between a size of a photoacoustic/ultrasound contrast agent and a frequency of an ultrasonic wave that is a photoacoustic signal emitted from the contrast agent. The diagnosis method is performed by using the diagnosis system. The diagnosis method includes: a photoacoustic signal generation step where light is emitted to the photoacoustic/ultrasound contrast agent such that the ultrasonic wave having a particular frequency is emitted; and a detection imaging step where the ultrasonic wave emitted at the photoacoustic signal generation step is detected and imaged. At the detection imaging step, the frequency of the ultrasonic wave, which differs depending on the size of the photoacoustic/ultrasound contrast agent and is emitted from the photoacoustic/ultrasound contrast agent, is selectively detected and imaged. The diagnosis method may further include an analysis step where the correlation between the size of the photoacoustic/ultrasound contrast agent and the frequency of the ultrasonic wave emitted from the contrast agent is analyzed.

At the photoacoustic signal generation step, light is emitted to the photoacoustic/ultrasound contrast agent such that the ultrasonic wave having a particular frequency is emitted, and the frequency of the ultrasonic wave emitted from the contrast agent differs depending on the size of the photoacoustic/ultrasound contrast agent. When a plurality of photoacoustic/ultrasound contrast agents having different sizes are used at the photoacoustic signal generation step, a plurality of ultrasonic waves having different frequencies are emitted.

At the detection imaging step, the ultrasonic wave emitted at the photoacoustic signal generation step is detected and imaged. The frequency of the ultrasonic wave, which differs depending on the size of the photoacoustic/ultrasound contrast agent and is emitted from the photoacoustic/ultrasound contrast agent, is selectively detected and imaged. When a plurality of photoacoustic/ultrasound contrast agents having different sizes are used and a plurality of ultrasonic waves having different frequencies are emitted at the photoacoustic signal generation step, a plurality of ultrasonic waves having different frequencies are respectively detected and imaged such that a plurality of images is obtained at the detection imaging step.

Hereinafter, the present invention will be described in detail with reference to experiments. However, the experiments are only for the purpose of describing the present invention in detail, and the scope and spirit of the present invention are not limited to the experiments.

### <Experiment 1> Formation of Porphyrin-Lipid that is a Combination of Porphyrin and Lipid

Porphyrin-lipid which was a subunit of porphyrin micro-bubbles was formed by acylation reaction of lysophosphatidylcholine and pyropheophorbide. First, 1-palmitoyl-2-hydroxyl-sn-glycero-3-phosphocholine of 100 nmol, pyropheophorbide of 50 nmol, 1-ethyl-3-(3-dimethylaminopropyl) carbodiimide of 50 nmol, 4-(dimethylamino) pyridine of 25 nmol, and N,N-diisopropylethylamine of 50 μL were dissolved in anhydrous dichloromethane of 10 mL, and were reacted in an argon environment for 48 hours in a shaded state at room temperature. Next, all solvents were evaporated and the residue was refined by thin layer chromatography (20×20 cm pre-coated silica gel plate with fluorescent indicator, 1.5 mm thickness). Here, refinement was performed with a retardation factor (Rf) of thin layer chromatography of 0.4 as a main band. The refinement method was that chromatography was performed by using diol modified silica, impurities were removed with dichloromethane containing methanol of 2% and %, and refinement was performed with dichloromethane containing methanol of 8%. The refined pyropheophorbide-lipid was aliquoted at a concentration of 1 μmol, and was dried by flowing nitrogen gas in argon environment at −20° C. The purity of extracted porphyrin-lipid was analyzed by high performance liquid chromatography and mass spectrometry (condition: Phenomenex Jupiter C4 column, 0.4 mL/min flow from % to 95% acetonitrile followed by hold 0.1% trifluoroacetic acid, compound eluted at 32 min, observed mass: 1013.1).

### <Experiment 2> Formation of a Photoacoustic/Ultrasound Contrast Agent (Porphyrin Micro-Bubble (MBs))

1) Porphyrin-lipid formed in the experiment 1 and 1,2-distearoyl-sn-glycero-3-phosphocholine (DSPC), which is a kind of phospholipid, were individually dissolved in chloroform that was a solvent.

2) Porphyrin-lipid and DSPC dissolved in chloroform were mixed in a sterile vial to respectively be 96 nM and 480 nM.

3) Next, after forming a lipid thin film by flowing nitrogen gas to evaporate chloroform, chloroform was completely evaporated by being stored in a vacuum state for at least 30 minutes. Phosphate buffer saline (PBS) of 990 μL and polyethylene-40-stearate (PEG40s) of 10 μL, which was an emulsifier, were added. Here, PEG40s had a preset concentration (2 mg/mL) such that the final mol ratio could be 10%.

4) In order to disperse a porphyrin-lipid thin film formed on an inner wall of the vial, the vial was immersed in water of 70° C. for one minute and at least a phase transition temperature (55-60° C.) of porphyrin lipid was maintained. The porphyrin-lipid thin film was dispersed through sonication for 30 seconds by using a bath sonicator (40 kHz). In order to completely disperse and uniformize the porphyrin-lipid film, the above-described procedure was repeated three times, whereby porphyrin liposome (sample 1) was completed.

5) Next, in order to form porphyrin micro-bubbles filled with perfluorocarbon gas (perfluoropropane gas in the experiment), first, a vial with the sample 1 of 5 mL was immersed in a water bath maintained to 70° C. to maintain the temperature, and was applied to a pin-type ultra sonicator (UP400s, Hielscher, Teltow, Germany) to fill perfluoropropane gas therein. The fill method with gas was flowing perfluoropropane gas to the end of the pin of the ultra sonicator, and locating the end of the pin near the surface of the sample 1, and performing ultra sonication on the sample 1 maintained to 70° C., whereby porphyrin micro-bubbles 1 to 3 (samples 2 to 4) filled with perfluoropropane gas were formed.

6) Size of porphyrin micro-bubbles 1 to 3 (samples 2 to 4) might be adjusted by a diameter of a tube generating gas located at the pin of the ultra sonicator and by intensity of sonication. In a case of porphyrin micro-bubbles 1 (sample 2), a tube having a diameter of about 0.52 mm was used to discharge perfluoropropane gas, the amplitude and cycle were respectively set to 100% and 1, and sonication was performed for one minute. In a case of porphyrin micro-bubbles 2 (sample 3), a tube having a diameter of 0.83 mm was used and sonication was performed for 30 seconds. Here, experiment values, the amplitude and cycle were set to 100% and 1. In a case of porphyrin micro-bubbles 3 (sample 4), a tube having a diameter of 0.83 mm was used as above and sonication was performed for 10 second, and used set values were the amplitude of 80% and cycle of 1.

### <Experiment 3> Analysis of Size (Diameter) Distribution of a Photoacoustic/Ultrasound Contrast Agent

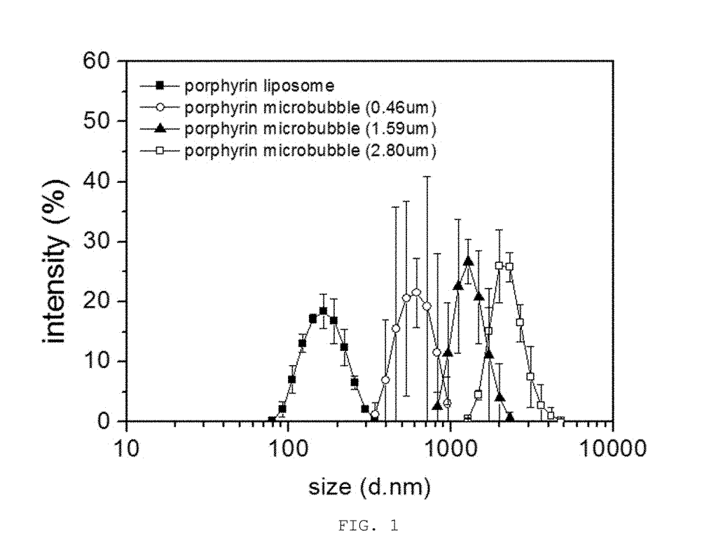

1) Size distributions of samples 1 to 4 were measured by using a dynamic light scattering method as shown in FIG. 1.

2) Referring to FIG. 1, size distributions of porphyrin liposome and porphyrin micro-bubbles 1, 2, and 3 were respectively 216.07±22.18 nm, 0.46±28.73 nm, 1.59±0.62 nm, and 2.80±0.35

### <Experiment 4> Analysis of Detection Frequency Band Distribution of an Ultrasound Transducer and Intensity of an Optimum Frequency and a Signal According to a Size of a Photoacoustic/Ultrasound Contrast Agent

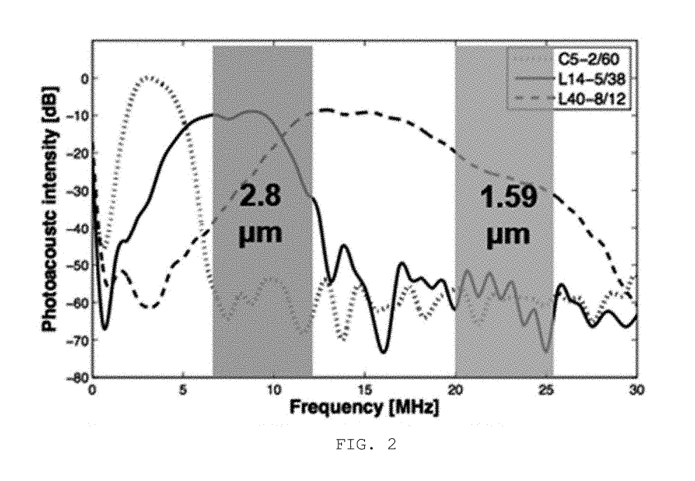

1) Respective detectable frequency bands of the ultrasound transducers C5-2/60, L14-5/38, and L40-8/12 used to detect photoacoustic signals emitted from the photoacoustic/ultrasound contrast agents were measured within a range of 1 to 30 MHz as shown in FIG. 2. In order to minimize overlap of detectable frequency ranges of the ultrasound transducers, detection bandwidth was set. In order to compensate for the difference in intensity of the signal that might occur due to the difference in detection performance between the ultrasound transducers, intensity of the signal was quantified to set a compensation value for the difference in intensity of the signal between the ultrasound transducers. Detection of the signal was performed through a general pulse-echo test, and the signal was emitted and detected by using the commercialized pulser-receiver and digital oscilloscope.

2) The optimum frequency for size distribution of the porphyrin micro-bubbles was calculated by the following formula 1 capable of calculating an ultrasound resonant frequency, and was derived by putting parameter values of porphyrin micro-bubbles 1 to 3 therein as shown in FIG. 2.

$$\begin{matrix}Formula 1 & \\ {f}_{res} = \frac{1}{2 π} \sqrt{\frac{3 {p}_{0}}{ρ {r}^{2}} + \frac{2 {S}_{p}}{ρ {r}^{3}}} & [ formula 1 ]\end{matrix}$$

(here, γ: polytropic gas index (1.06), p0: ambient pressure (1000 kgm-3), ρ: density of the medium (100 kPa), r: radius of the porphyrin-micro-bubble, Sp: shell stiffness (5.32±0.43 N/m)).

3) Respective detectable frequency bands of ultrasound transducers C5-2/60, L14-5/38, and L40-8/12 were verified as 1.8-5.1, 4.0-11.0, and 9.6-20.2 MHz. The optimum resonant frequencies of the samples 2 to 4 were respectively calculated as 100 MHz, 23 MHz, and 10 MHz. Therefore, it was derived that the ultrasound transducers L40-8/12 and L14-5/38 were respectively optimum for samples 3 and 4.

### <Experiment 5> Photoacoustic Image Measurement

1) In order to obtain photoacoustic images of samples 1 to 4, Q-switch Nd: YAG laser excitation system (Surelite III-10 and Surelite OPO Plus, Continuum Inc., Santa Clara, Calif., USA), to which a function generator (AFG3252, Tektronix Corp., Beaverton, Oreg., USA) was connected, was used to provide light energy, and a commercialized ultrasound imaging device was used to obtain the images. The light source emitting light to samples 1 to 4 used a wavelength of 700 nm that might optimally absorbed by porphyrin, a pulse ratio of 10 Hz, and intensity of 5 mJ/cm2. Photoacoustic signals occurring due to the samples 1 to 4 were obtained by using the ultrasound transducers C5-2/60, L14-5/38, and L40-8/12 provided with the ultrasound imaging device. The obtained signals were reconstructed and quantified by using MATLAB code. In order to obtain images, tygon tubes, unaffected by the light source, were positioned in the center of a water tank. After filling the water tank with water that is a medium through which the ultrasonic wave could propagate, the light source and the ultrasound transducers were positioned at a distance where the light could be focused. Next, samples 1 to 4 were respectively administered to the tygon tubes, and photoacoustic signals were obtained by respective ultrasound transducers. Among the obtained signals, only signals for the previously verified detection frequency bands of respective ultrasound transducers were imaged, and intensity of the signal was quantified.

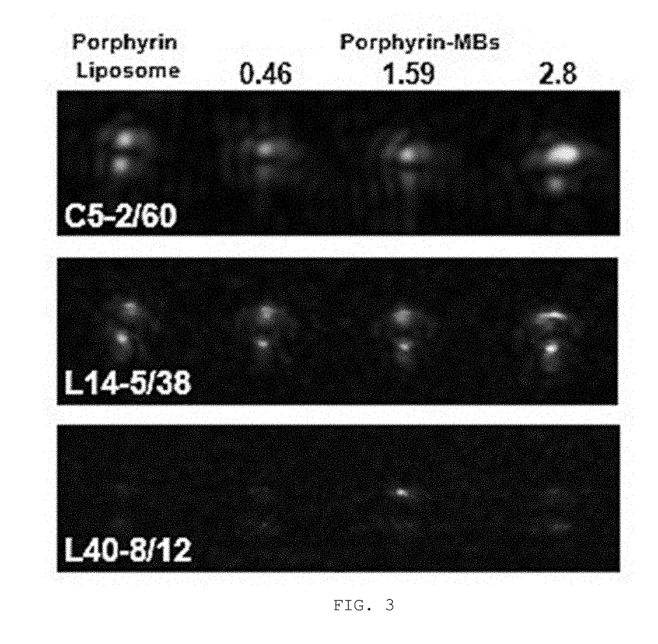

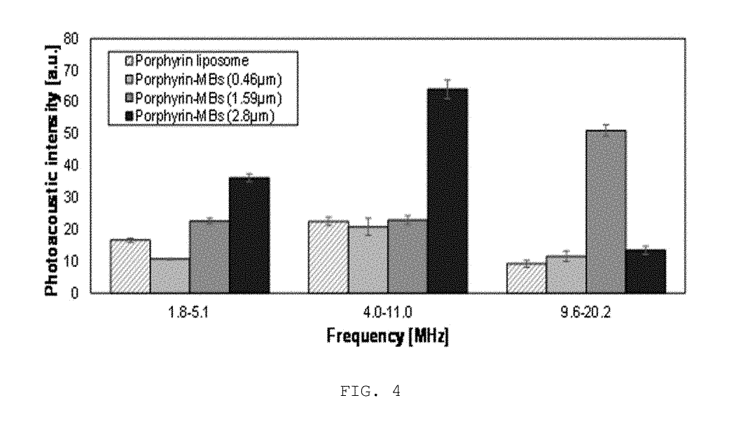

2) Photoacoustic signals of the samples 1 to 4 were detected by respective ultrasound transducers, and the result of images obtained by using these and intensity of the signals were shown in FIGS. 3 and 4. Referring to FIGS. 3 and 4, in the frequency band of 1.8-5.1 MHz detectable by the ultrasound transducer C5-2/60, the contrast effect was not excellent in the samples 2 and 3, but the contrast effect was excellent in the sample 4 having the most similar resonant frequency. Particularly, in the ultrasound transducer L14-5/38 having the optimum resonant frequency of the sample 4, intensity of images of the samples 2 and 3 were similarly maintained. In contrast, intensity of the image of the sample was dramatically increased, and had about three times difference in intensity of the image, compared to photoacoustic signals of the remaining contrast agents (samples 2 and 3). Also, in the ultrasound transducer L40-8/12 having a frequency band similar to the optimum resonant frequency of the sample 3, intensity of the image of the sample 3 was selectively amplified. Accordingly, by using resonance caused by fusion of a photoacoustic contrast agent (role of porphyrin in the present invention) generally having a wide frequency band and an ultrasound contrast agent (role of the micro-bubbles consisting of gas-filled lipids, etc. in the present invention), the signal may be selectively amplified in a particular frequency. Also, the desired optimum resonant frequency band may be selectively adjusted by adjusting the size of the porphyrin micro-bubble.

### <Experiment 6> Resonant Frequency Analysis of a Photoacoustic/Ultrasound Contrast Agent

1) A resonant frequency of the photoacoustic/ultrasound contrast agent was analyzed to demonstrate that a photoacoustic signal was emitted from the photoacoustic/ultrasound contrast agent due to resonance with the contrast agent.

2) First, the photoacoustic/ultrasound contrast agent (sample 5) representing the strongest signal in the ultrasound transducer (L14-5/38) having a frequency band of 4.0-11.0 MHz and the photoacoustic/ultrasound contrast agent (sample 6) representing the strongest signal in the ultrasound transducer (L40-8/12) having a frequency band of 9.6-20.2 MHz were prepared by using a formation method described in the experiment 2. The formed samples 5 and 6 respectively had size distributions of 3.17±0.53 μm and 1.92±0.21 μm.

3) Respective optimum resonant frequencies of the photoacoustic/ultrasound contrast agents of the samples 5 and 6 were theoretically calculated based on formula 1. Next, in order to experimentally verify resonant frequencies of the photoacoustic/ultrasound contrast agents of the samples 5 and 6, signals of the ultrasonic wave were analyzed at intervals of 0.5 MHz in the frequency band of 4-20 MHz. A single element transducer capable of emitting 4-15 MHz and 10-20 MHz was used to emit the ultrasonic wave. In order to detect the signal of the emitted ultrasonic wave, signals of ultrasonic waves of all frequencies were detected by using a hydrophone capable of detecting the ultrasonic waves of all frequencies, and were quantitatively analyzed. In order to adjust the intensities of the ultrasonic waves emitted from respective frequencies to the same level, intensity of the emitted ultrasonic wave was measured and adjusted to the same level at intervals of 0.5 MHz. In order to detect the signal of the ultrasonic wave caused by resonance with the photoacoustic/ultrasound contrast agent, the agarose phantom was located between the single element transducer and the hydrophone, and the signal of the ultrasonic wave emitted due to resonance with the ultrasonic wave by injecting the photoacoustic/ultrasound contrast agent into a body was detected and quantitatively analyzed.

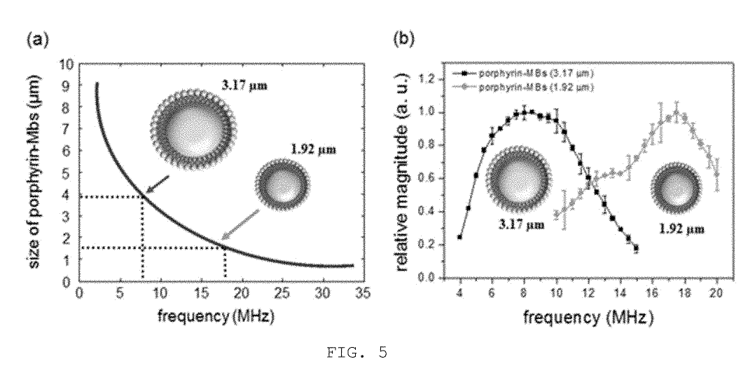

2) According to theoretical resonant frequencies of the photoacoustic/ultrasound contrast agents of the samples 5 and 6, the sample 5 had a resonant frequency band of 15.2-21.0 MHz and the optimum resonant frequency of 17.7 MHz. Also, the sample 6 had a resonant frequency band of 6.84-11.4 MHz and the optimum resonant frequency of 8.62 MHz. FIG. 5(a) is a graph illustrating a theoretical optimum resonant frequency band according to the size of the photoacoustic/ultrasound contrast agent. FIG. 5(b) is a graph experimentally illustrating optimum resonant frequencies of the samples 5 and 6. Theoretical and experimental results show similar result that corresponds to the frequency band where the strongest photoacoustic signal was detected as shown in FIG. 4. Based on the results in FIGS. 4 and 5, the photoacoustic signal is emitted due to resonance with the photoacoustic/ultrasound contrast agent, and the frequency band of the photoacoustic signal may be unrestrainedly adjusted. Multi-imaging may be performed by using an ultrasound transducer capable of detecting a particular frequency of the photoacoustic signal and through signal processing of the ultrasound imaging device.

Although preferred embodiments of the present invention have been described above, the scope of the present invention is not limited to the embodiments, and changes or modifications from the spirit of the present invention defined in the following claims by those skilled in the art are also included in the scope of the present invention.

1. A diagnosis method using a photoacoustic/ultrasound contrast agent, the method comprising:
obtaining a photoacoustic image by considering a correlation between a size of a photoacoustic/ultrasound contrast agent and a frequency of an ultrasonic wave that is a photoacoustic signal emitted from the contrast agent.

2. The diagnosis method of claim 1, further comprising:
emitting, at a photoacoustic signal generation step, light to the photoacoustic/ultrasound contrast agent such that the ultrasonic wave having a particular frequency is emitted; and
detecting, at a detection imaging step, the ultrasonic wave emitted at the photoacoustic signal generation step and imaging the ultrasonic wave,
wherein at the detection imaging step, the frequency of the ultrasonic wave that varies depending on the size of the photoacoustic/ultrasound contrast agent is selectively detected and imaged.

3. The diagnosis method of claim 2, wherein at the photoacoustic signal generation step, a plurality of photoacoustic/ultrasound contrast agents having different sizes is used such that a plurality of ultrasonic waves having different frequencies is emitted, and
at the detection imaging step, the plurality of ultrasonic waves having the different frequencies is detected and imaged such that a plurality of photoacoustic images is obtained.

4. The diagnosis method of claim 3, wherein the plurality of photoacoustic/ultrasound contrast agents is injected into different parts in a body, and
at the detection imaging step, the plurality of photoacoustic images of the different parts is simultaneously obtained.

5. The diagnosis method of claim 3, wherein the plurality of photoacoustic/ultrasound contrast agents is injected into a same part in a body, and
at the detection imaging step, the plurality of photoacoustic images of the same part is obtained.

6. The diagnosis method of claim 3, wherein the plurality of photoacoustic/ultrasound contrast agents targets different biomarkers such that the plurality of photoacoustic images is obtained at the detection imaging step, whereby multi-imaging is performed.

7. The diagnosis method of claim 6, wherein each of the plurality of photoacoustic/ultrasound contrast agents includes a ligand that is conjugated to a surface thereof and targets a particular biomarker.

8. The diagnosis method of claim 6, wherein at the detection imaging step, the plurality of photoacoustic images has different colors.

9. A diagnosis system using a photoacoustic/ultrasound contrast agent, the system obtaining a photoacoustic image by considering a correlation between a size of a photoacoustic/ultrasound contrast agent and a frequency of an ultrasonic wave that is a photoacoustic signal emitted from the contrast agent

10. The diagnosis system of claim 9, further comprising:
the photoacoustic/ultrasound contrast agent being injected into a body and being used as a photoacoustic and ultrasound contrast agent;
a light emitting device emitting light to the photoacoustic/ultrasound contrast agent; and
a detection imaging device detecting the ultrasonic wave, emitted from the photoacoustic/ultrasound contrast agent by absorbing the light of the light emitting device, and imaging the ultrasonic wave,
wherein the detection imaging device selectively detects the frequency of the ultrasonic wave that varies depending on the size of the photoacoustic/ultrasound contrast agent so as to image the frequency.

11. The diagnosis system of claim 10, wherein the detection imaging device includes an ultrasound transducer detecting the ultrasonic wave emitted from the photoacoustic/ultrasound contrast agent, and
the ultrasound transducer has a detection frequency band containing the frequency of the ultrasonic wave emitted from a particular contrast agent.

12. The diagnosis system of claim 11, wherein a plurality of photoacoustic/ultrasound contrast agents having different sizes is used such that frequencies of ultrasonic waves emitted from the plurality of photoacoustic/ultrasound contrast agents are different, and
the detection imaging device obtains a plurality of photoacoustic images by individually detecting and imaging the different frequencies of the ultrasonic waves.

13. The diagnosis system of claim 12, wherein the detection imaging device includes a plurality of ultrasound transducers detecting the ultrasonic waves emitted from the plurality of photoacoustic/ultrasound contrast agents, and
each of the plurality of ultrasound transducers has a frequency band containing the frequency of the ultrasonic wave emitted from the associated contrast agent.

14. The diagnosis system of claim 12, wherein as the ultrasound transducer, one wideband ultrasound transducer having all frequency bands of the ultrasonic waves emitted from the plurality of photoacoustic/ultrasound contrast agents is used, or an integrated ultrasound transducer having different frequency bands is used.

15. The diagnosis system of claim 9, wherein the photoacoustic/ultrasound contrast agent includes micro-bubbles having gas inside thereof and a photoacoustic contrast component that is conjugated to a surface of the micro-bubbles or is stacked inside thereof.

16. The diagnosis system of claim 15, wherein the photoacoustic/ultrasound contrast agent includes a ligand that is conjugated to the surface of the micro-bubbles and targets a particular biomarker,
the gas is at least one of perfluorocarbon and sulfur hexafluoride,
the micro-bubbles are made of protein or lipid, and
the photoacoustic contrast component is at least one of porphyrin, indocyanine green, green fluorescence protein (GFP), ferritin, and gold nanorod.

17. The diagnosis system of claim 15, wherein the frequency of the ultrasonic wave emitted from the photoacoustic/ultrasound contrast agent is a resonant frequency that occurs when the ultrasonic wave emitted from the photoacoustic contrast component is resonated by the micro-bubble.

18. The diagnosis system of claim 10, wherein the photoacoustic/ultrasound contrast agent includes micro-bubbles having gas inside thereof and a photoacoustic contrast component that is conjugated to a surface of the micro-bubbles or is stacked inside thereof.

19. The diagnosis system of claim 11, wherein the photoacoustic/ultrasound contrast agent includes micro-bubbles having gas inside thereof and a photoacoustic contrast component that is conjugated to a surface of the micro-bubbles or is stacked inside thereof.

20. The diagnosis system of claim 12, wherein the photoacoustic/ultrasound contrast agent includes micro-bubbles having gas inside thereof and a photoacoustic contrast component that is conjugated to a surface of the micro-bubbles or is stacked inside thereof.
